(19) United States
(12) Patent Application Publication
(10) Pub. No.: US 2026/0294016 A1
(43) Pub. Date: Oct. 1, 2026

(54) MONITORING SYSTEM FOR MEASURING VIBRATIONS

(71) Applicant: MS2PROTECT AB, Helsingborg (SE)

(72) Inventors: Niclas FOLKESSON, Helsingborg (SE); Jan SAMUELSSON, Helsingborg (SE)

(21) Appl. No.: 19/477,303

(22) PCT Filed: Apr. 26, 2024
(86) PCT No.: PCT/EP2024/061582

§ 371 Date: Oct. 21, 2025

(30) Foreign Application Priority Data
Apr. 26, 2023 (SE) 2350503-5

(51) Int. Cl. A41D 19/00; A41F 1/06; G01H 1/00; G08B 6/00; G08B 21/18

(52) U.S. Cl. CPC A41D 19/0027; A41D 19/0013; A41D 19/0048; A41F 1/06; G01H 1/00; G08B 6/00; G08B 21/182; A41D 2600/20

(57) ABSTRACT

A system for measuring vibrations transmitted to a hand (1) of a user comprises a housing (2) enclosing a vibration monitor. The system further comprises a holding glove element (3) for at least an index finger and a thumb, the glove element further comprising a pocket (4) configured to hold the housing (2) over the first dorsal interossei muscles on the back side of the hand (1) of a user.

### TECHNICAL FIELD

The present invention relates to a vibration monitor device which is configured to be releasably attached to a hand of a user, comprising a housing enclosing a vibration monitor.

### BACKGROUND

Hand held and hand guided powers tools usually transmit vibration to the hands and arms of a power tool operator. It is known that such transmitted vibration, which is often termed Hand Arm Vibration (HAV), can lead to painful and disabling disease as a consequence of long-term exposure. Regulating the exposure of potentially damaging hand-arm vibrations to machine operators requires affordable and easy-to-use personalized devices which will measure and record the cumulative exposure of vibrations to an operator during the course of a working period.

A directive acknowledges the possible damaging consequences of vibration for human health and lays down maximum levels of vibration exposure to avoid “white finger”. White finger is a medical condition of numbness or pain that arises from continuous use of vibrating tools. In extreme cases white finger can lead to loss of one or more digits. Other issues from exposure to vibrations include, for instance, carpal tunnel syndrome.

The directive lays down a careful specification of what cumulative vibration levels the user is allowed to be exposed to. Above this specified dosage work must stop for that day.

Vibration is caused by a moving object. Two important physical characteristics when evaluating the risk of health effects or discomfort associated with exposure to vibration are acceleration and frequency.

In basic terms, acceleration, also known as the vibration level, is the measure of how fast the object is moving. The faster the object moves, the higher the acceleration or vibration level. In terms of vibration, acceleration is measured in m/s².

In terms of frequency this can't be directly translated to vibration. Each part of the body is susceptible to a vibration of a different frequency. For example, legs are most susceptible to vibration of 2-20 Hz, the stomach to 4-8 Hz, the hand to 30-50 Hz, and the arm to 5-10 Hz. This means that exposure to vibrating objects that oscillate in the frequency range to which the hand and arm are most susceptible result in the highest risk of adverse health effects from HAV.

Per section 7.11(a) of the Occupational Health and Safety (OHS) Regulation, an employer must ensure that workers are not exposed to HAV in excess of the limits specified in the American Conference of Governmental Industrial Hygienists (ACGIH) publication, Threshold Limit Values and Biological Exposure Indices. In the summary of WorkSafeBC vibration exposure limits, with a daily exposure duration of 8 hours, the vibration exposure limit is 5 m/s².

The 5.0 m/s² exposure limit is the maximum vibration level that a worker can be exposed to averaged over the course of an 8-hour shift. This means that a worker may be exposed to vibration in excess of 5.0 m/s² as long as the duration of exposure is less than 8 hours. For example, a worker can operate a tool that exposes them to a vibration level of 10.0 m/s² for less than half of the time of an 8-hour shift. If the worker doesn't operate any more tools that result in vibration exposure during the remainder of their shift, they will not have been overexposed to vibration.

The 5.0 m/s² exposure limit does not represent a boundary between safe and unsafe exposure and therefore, it is good practice and more protective for workers to take precautions before exposure reaches the exposure limit. This can be done by using 2.5 m/s² as a guideline vibration level.

What the directive does not do is suggest how this exposure is to be measured. Technically, vibration measurements can be made with high quality but relatively expensive hardware that exists on the market today. This hardware will have been used in research work involved in the definition of the standards.

EP 3 799 582 A1 discloses a vibration monitor which is releasably attached to an arm or hand of an operator during use of a power tool. The vibration monitor comprises a vibration sensor and a processor. The vibration sensor senses vibration sustained by the arm or hand of the operator when the vibration monitor is attached to the arm or hand of the operator and provides: a first vibration signal corresponding to vibration in a first axis; a second vibration signal corresponding to vibration in a second axis; and a third vibration signal corresponding to vibration in a third axis.

There is thus a need to provide a vibration dosage meter that is light, unobtrusive, comfortable, and easy to use even when wearing work gloves and preferably cheap enough for every worker to have one. It should calculate the vibration dose accurately and provide a clear indication of when the dosage limit has been reached. Also, the fine motor skills should not be affected by wearing this kind of device. The group of workers that could benefit from such a device comprises, for instance, various craftsmen, healthcare staff, e.g., dentists, gardeners, city staff, workers within transport etc.

### SUMMARY

It is therefore an object of the present invention to provide a system with a vibration monitor device that is easy to use and unobtrusive.

According to a first aspect of the present disclosure a system for measuring vibrations transmitted to a hand of a user comprises a housing enclosing a vibration monitor. The system further comprises a holding glove element for at least an index finger and a thumb and the glove element further comprising a pocket configured to hold the housing over the first dorsal interossei muscles on the back side of the hand of a user. According to one aspect the glove element is a glove. According to a further aspect, the glove element is a fingerless glove element. An advantage with a fingerless glove element is that it is less invasive for a user when handling a tool. However, in some instances a regular glove is needed anyway and a glove with a pocket for the housing could then be used as long as the pocket and thus the housing is arranged over the first dorsal interossei muscles on the back side of the hand.

In the context of the invention, the vibration monitor comprises at least a control unit and a vibration sensor.

By placing the housing with the vibration monitor over the first dorsal interossei muscles on the back of the hand, the device is both minimally obtrusive and effective in measuring the vibrations. With this configuration the housing with the vibration monitor will be out of the way for a user who can perform as normal with maintained control of whatever tool is used at the moment. Not placing the vibration monitor on the palm of the hand but rather on the back of a hand means that the vibrations are slightly dampened, assuming that a power tool is in contact with the palm of the hand. In order to detect the correct vibration level, the vibration monitor is preferably calibrated to imitate the vibration that the palm of the hand has been subjected to.

Preferably, the glove element further comprises a wrist strap for holding the system secured to the hand of a user.

According to one aspect of the present disclosure the external layer of the pocket is elastic. Thus, the housing could be held in the correct position and is easily inserted and removed. Another option is to have a pocket where the opening could be closed by a Velcro band.

According to a further aspect of the present disclosure, the housing further comprises a transmitter connected to the vibration monitor. The transmitter is arranged to wirelessly interact with an application in a remote device, such as a mobile phone, for registering vibration data corresponding to the vibrations a user are exposed to. The vibration data collected may then be displayed in the remote device for checking whether or not a threshold is passed or not. According to an alternative aspect of the present disclosure, the system further comprises a receiver for receiving input such as threshold level of vibrations, i.e., setting the threshold for when there may be a health risk continuing working.

According to yet a further aspect of the present disclosure the application is arranged to provide a notification to the user when a predetermined level of accumulated vibrations have been registered. In this way, the user is immediately notified. The set level could for instance be at 80% of the allowed level of vibrations for a day in order for the user to be able to plan when to conclude the work.

According to an alternative aspect of the present disclosure the application of the mobile device has a connection with a computer data network. According to some legislation, an employer might be expected to keep track of the working environment for the employees and may with such a connection monitor from a distance the exposure to vibrations of the employees. Also, in a company with several coworkers, a person managing the planning of the tasks to be performed could thus in this way plan the work of the coworkers to make sure that no one is subjected to a too large level of vibrations.

The vibration monitor according to an advantageous aspect of the present disclosure further comprises a haptic generation module arranged to produce a signal upon reaching a predetermined threshold level of vibrations. Having the housing arranged over the first dorsal interossei muscles on the back side of the hand provides a better chance for the user to notice the signal. For instance, a vibration signal could be difficult to separate from the vibrations of a power tool should the housing of the vibration monitoring device be arranged on the palm of the hand. The same applies to a light signal which could be easily detected according to the device of the present disclosure compared to a palm placed monitor where the light might not even be visible. If the device is equipped with a receiver, a threshold level could be set for when said haptic signals are to be produced.

According to a further aspect of the present disclosure, the system further comprises one or any combination of sensors from the group comprising; motion sensor, temperature sensor, gas sensor, sound sensor, particle sensor, and light sensor. As previously mentioned, having the housing of the monitoring device over the first dorsal interossei muscles on the back side of the hand provides for easier detection of a haptic signal. A further advantage of the placement is when the system is combined with another sensor, i.e., one of the sensors mentioned above. For instance, a sound sensor placed over the first dorsal interossei muscles on the back side of the hand will be exposed basically to the same level of sound as a sound sensor separately placed elsewhere on a user. Another possibility would be to have the system on the hand combined with a sensor in wireless communication with each other. For instance, a temperature sensor could be placed on a helmet of a user.

According to yet another aspect of the present disclosure the vibration monitor is adapted to measure vibrations from about 2 Hz and up.

Further features of, and advantages with, the present invention will become apparent when studying the appended claims and the following description. The skilled person realize that different features of the present invention may be combined to create embodiments other than those described in the following, without departing from the scope of the present invention.

### BRIEF DESCRIPTION OF THE DRAWINGS

The above, as well as additional objects, features and advantages of the present invention, will be better understood through the following illustrative and non-limiting detailed description of exemplary embodiment of the present invention, wherein

FIG. 1 is a view of a back side of a hand with an embodiment of a system according to the present disclosure, and

FIG. 2 shows the system of FIG. 1 with a remote device.

### DETAILED DESCRIPTION OF EXAMPLE EMBODIMENTS OF THE INVENTION

The present invention will now be described more fully hereinafter with reference to the accompanying drawings, in which exemplary embodiments of the invention are shown. The invention may, however, be embodied in many different forms and should not be construed as limited to the embodiments set forth herein; rather, these embodiments are provided for thoroughness and completeness. Like reference character refer to like elements throughout the description.

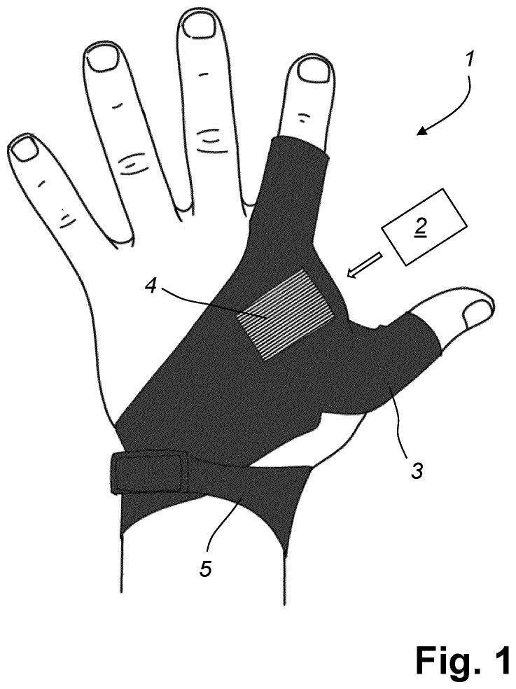

With reference to FIG. 1, a system for measuring vibrations transmitted to a hand 1 of a user comprising a housing 2 enclosing a vibration monitor is shown. The system shown in the figure further comprises a fingerless holding glove element 3 for an index finger and a thumb wherein the glove element further comprising a pocket 4 configured to hold the housing 2, shown outside the pocket, over the first dorsal interossei muscles on the back side of the hand 1 of a user. Thus, the system is arranged to hold the vibration monitor/housing 2 over the soft parts of the hand between the thumb and the index finger.

The system is shown with the housing 2 to be inserted in the pocket 4 of the holding glove element 3.

As also can be seen glove element 3 further comprises a wrist strap 5. Using the strap 5, the glove element 3 will stay on the hand 1 when pulled out of a working protection glove or similar. The strap 5 could be attached by for instance a Velcro band.

Further, the glove element 3 could be made to fit either a right hand or a left hand. Under certain circumstances a system according to the present invention could be worn on both the left and the right hand. Also, depending to some extent on the material of the glove element 3, it could be turned inside out, i.e., the same glove element 3 could be used for either the left or the right hand.

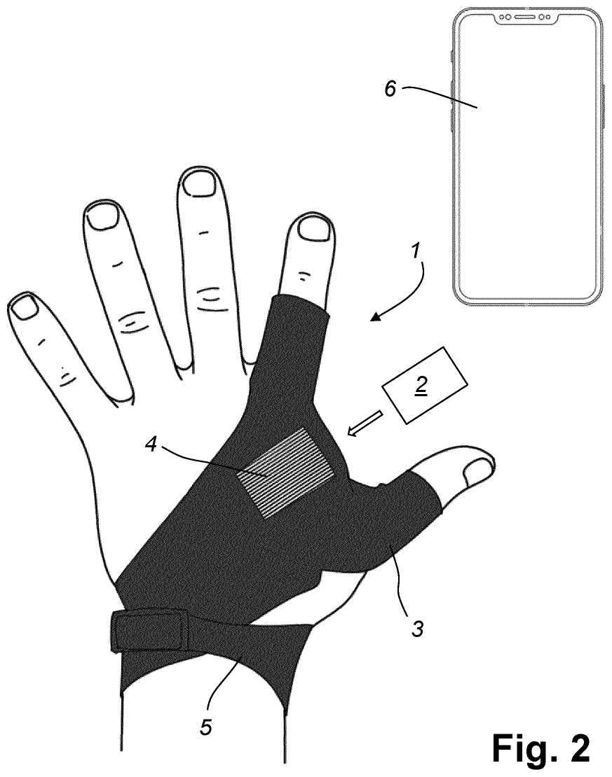

FIG. 2 shows the system of FIG. 1 with a remote device 6, in this specific case a mobile phone. Hence, the remote device 6 has an application that via the mobile phone/remote device 6 has a wireless connection to the housing 2 with the vibration monitor.

While one embodiment of the present invention has been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the claims. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used.

The indefinite articles “a” and “an,” as used herein in the specification and in the claims, unless clearly indicated to the contrary, should be understood to mean “at least one.” The phrase “and/or,” as used herein in the specification and in the claims, should be understood to mean “either or both” of the elements so conjoined, i.e., elements that are conjunctively present in some cases and disjunctively present in other cases.

1-9. (canceled)

10. A system for measuring vibrations, the system comprising:
a housing enclosing a vibration monitor;
a holding glove for at least an index finger and a thumb, wherein the holding glove comprises a pocket configured to hold the housing over the first dorsal interossei muscles on a back side of a hand of a user.

11. The system according to claim 10, wherein the holding glove further comprises a wrist strap.

12. The system according to claim 10, wherein the pocket comprises an elastic outer layer.

13. The system according to claim 10, further comprising a transmitter connected to the vibration monitor.

14. The system according to claim 13, wherein the system further comprises a remote device with a software application, wherein the transmitter is configured to wirelessly interact with the software application in the remote device for recording vibration data corresponding to the vibrations a user is exposed to.

15. The system according to claim 14, wherein the remote device comprises a mobile phone.

16. The system according to claim 14, further comprising a receiver.

17. The system according to claim 10, wherein the system is configured to provide a notification to the user when a predetermined level of accumulated vibrations have been sensed by the vibration monitor.

18. The system according to claim 14, wherein the software application has a connection with a computer data network.

19. The system according to claim 15, wherein the software application has a connection with a computer data network.

20. The system according to claim 10, wherein the system further comprises a haptic generator configured to produce a signal when a predetermined threshold level of vibrations is sensed.

21. The system according to claim 10, further comprising any one or more of a motion sensor, a temperature sensor, a gas sensor, a sound sensor, a particle sensor, and a light sensor.

22. The system according to claim 10, wherein the vibration monitor is configured to measure vibrations equal to or greater than 2 Hz.
